(19) United States
(12) Patent Application Publication
(10) Pub. No.: US 2022/0068433 A1
(43) Pub. Date: Mar. 3, 2022

(54) COMPUTATIONAL DETECTION OF COPY NUMBER VARIATION AT A LOCUS IN THE ABSENCE OF DIRECT MEASUREMENT OF THE LOCUS

(71) Applicant: GUARDANT HEALTH, INC., Redwood City, CA (US)

(72) Inventor: Stephen FAIRCLOUGH, Redwood City, CA (US)

(21) Appl. No.: 17/459,743

(22) Filed: Aug. 27, 2021

Related U.S. Application Data
(60) Provisional application No. 63/071,206, filed on Aug. 27, 2020.

(51) Int. Cl. G16B 20/10

(52) U.S. Cl. CPC G16B 20/10

(57) ABSTRACT

Methods and systems are described for improving detection of copy number loss of a locus of interest without requiring direct measurement of the locus of interest. The locus of interest may include the human leukocyte antigen (HLA) locus, for which copy number loss is implicated in cancer. Direct measurement of the HLA locus, which is on human chromosome 6, may be unavailable due to the polymorphic nature of the HLA. Thus, the system may infer the genetic state of the HLA locus. For example, the system may generate and use a probabilistic ML model that determines a probability that a given sample of a subject has a copy number loss at the HLA locus based on copy number determinations of segments of chromosome 6 that are determined to be predictive of copy number loss at the HLA locus.

### RELATED APPLICATIONS

This application claims the benefit of U.S. Provisional Application No. 63/071,206, filed on Aug. 27, 2020, which is incorporated herein by reference in its entirety.

### BACKGROUND

Genetic variants, such as insertions, deletions, substitutions, rearrangements and copy number variants may be correlated with disease and response to therapeutic intervention. Identifying genetic variants accurately is therefore becoming increasingly important for diagnosing and treating disease.

One example of a clinically important genetic variant is a copy number loss that results in a loss of function. For example, a copy number loss relating to the human leukocyte antigen (HLA) may facilitate immune evasion, which is a hallmark of cancer. Thus, it may be important to detect copy number loss relating to HLA. However, because of the polymorphic nature of the HLA locus, it is frequently excluded from target capture methods and copy number analysis. As a result, it may become computationally difficult to perform precision diagnostics on copy number loss for an HLA locus and other loci in which direct measurement of a locus is inaccurate or unavailable.

### SUMMARY

The disclosure relates to an improved computer technology, including probabilistic machine-learning (ML), that provides precision diagnosis based on detection of a genetic state of a locus of interest in genetic material without requiring direct measurement of the locus of interest. The genetic material may include Deoxyribonucleic Acid (DNA) or Ribonucleic Acid (RNA) from a genome, chromosome, or other genetic material of a sample. The genetic state may include a variation from a wildtype sequence of the nucleic acid sequenced from the sample. Such variation may include, without limitation, a copy number variant (CNV) (which may include a series of deletions also referred to as copy number loss (CNL) relative to the wildtype state or insertions), a rearrangement, and/or other states. Based on the diagnostic, one or more treatment options may be determined. Examples that follow will refer to detection of CNV at a locus of interest and in particular to CNL at the HLA locus. However, other types of genetic states of other loci of interest may be modeled.

Direct measurement of the HLA locus, which is on human chromosome 6, may be unavailable due to the polymorphic nature of the HLA. Thus, the system may infer the genetic state of the HLA locus. For example, the system may generate and use a probabilistic ML model that determines a probability that a given sample of a subject has a copy number loss at the HLA locus based on copy number determinations of segments of chromosome 6 that are determined to be predictive of copy number loss at the HLA locus.

In some examples, the disclosure relates to a computer system improved to determine a copy number variant (CNV) at a locus of interest in a sample of a subject when direct measurements of copy number at the locus of interest are unavailable. The computer system may include a processor programmed to access a plurality of sequence reads of a sample. The processor may map the plurality of sequence reads to a reference sequence, the reference sequence including a sequence of interest that includes the locus of interest. The processor may generate a copy number score indicating a copy number of regions along the sequence of interest, the copy number score not include a copy number at the locus of interest. The processor may generate a plurality of segments along the sequence of interest. The processor may for each segment of the plurality of segments: generate a respective segmented copy number score based on the segment and the copy number score, generate a respective weight based on historical performance of the segment in predicting a CNV at the locus of interest, and generate a weighted score based on the respective weight and the respective segmented copy number score. The processor may apply a probabilistic machine-learning (ML) copy number classifier to generate a probability of the CNV at the locus of interest based on an aggregate of the weighted score of each segment.

In some examples, the processor is further programmed to train the probabilistic ML copy number classifier based on a maximum likelihood estimation.

In some examples, the processor is further programmed to train the probabilistic ML copy number classifier based on a Hidden Markov Model. In some examples, the processor is further programmed to train the probabilistic ML copy number classifier based on Bayesian inference.

In some examples, the disclosure relates to a computer system to determine a genetic state of a locus of interest of a genetic material in a sample of a subject. The computer system may include a processor programmed to detect a genetic state associated with a segment in the genetic material in the sample. The processor may generate a probability that the locus of interest outside the segment is also in the genetic state. The processor may further predict a disease condition of the subject based on the generated probability.

In some examples, the genetic material may include a chromosome at which the locus of interest is located and the genetic state comprises a copy number variant at the segment of the chromosome.

In some examples, to generate the probability, the processor is further programmed to determine a level of deletion of the chromosome based on the copy number variant at the segment. In these examples, the generated probability rises as a proportional function with the level of deletion.

In some examples, to generate the probability, the processor is further programmed to determine a physical distance between the segment and the locus of interest. In these examples, the generated probability rises as an inversely proportional function with the physical distance.

In some examples, to detect the genetic state, the processor is programmed to: access sequence reads generated from the sample, and analyze sequence coverage information of the sequence reads.

In some examples, to generate the probability, the processor is programmed to generate the probability without sequence reads that cover the locus of interest.

In some examples, to generate the probability, the processor is programmed to generate the probability without genotyping data for the locus of interest.

In some examples, the locus of interest is associated with the disease condition.

In some examples, wherein the locus of interest comprises a Human Leukocyte Antigens (HLA) locus.

In some examples, one or more target genes in the locus of interest and one or more reference genes in the segment are not within the same gene regulation pathway.

In some examples, one or more target genes in the locus of interest are in genetic linkage with one or more reference genes in the segment. In these examples, the processor is programmed to identify one or more segments, including the segment, to analyze for detection of copy number variants based on the genetic linkage.

In some examples, the genetic state includes at least one of: a copy number variation, an aneuploidy, a segmental loss, a fusion, or an indel.

In some examples, the processor is further programmed to determine a treatment based on the predicted disease condition.

In some examples, to detect the genetic state, the processor is further programmed to determine that the segment includes a copy number loss based on probe data for probes directed to the segment. In these examples, to generate a probability that the locus of interest outside the segment is also in the genetic state, the processor is further programmed to determine that the locus of interest also includes a copy number loss.

In some examples, the disclosure relates to a computer system for predicting a disease state of an individual based on a predicted state of a locus of interest of genetic material of the individual. The computer system may include a processor programmed to access probe data for a plurality of segments in the genetic material. The plurality of segments may be distinct from the locus of interest. The processor may further determine whether or not each segment of the plurality of segments has a respective copy number loss based on the probe data. The processor may generate, without probe data of probes directed to the locus of interest, a probability that the locus of interest also includes a copy number loss based on the determination of whether or not each segment of the plurality of segments has a respective copy number loss. The processor may further predict a disease condition of the subject based on the determined probability.

In some examples, the disclosure relates to a computer system. The computer system may include a processor programmed to access probe data for probes directed to a segment. The processor may determine that a segment includes a first copy number loss mutation based on probe data for probes directed to the segment. The processor may further generate a probability that a locus of interest outside the segment includes a second copy number loss based on the determination that the segment includes the first copy number loss. The processor may predict a disease condition of the subject based on the determined probability.

In some examples, the disclosure relates to a method, implemented by a processor, of determining a copy number variant (CNV) at a locus of interest in a sample of a subject when direct measurements of copy number at the locus of interest are unavailable. The method may include accessing, by the processor, a plurality of sequence reads of a sample. The method may further include mapping, by the processor, the plurality of sequence reads to a reference sequence, the reference sequence including a sequence of interest that includes the locus of interest, generating, by the processor, a copy number score indicating a copy number of regions along the sequence of interest, the copy number score not include a copy number at the locus of interest, generating, by the processor, a plurality of segments along the sequence of interest. The method may further include, for each segment of the plurality of segments: generating, by the processor, a respective segmented copy number score based on the segment and the copy number score, generating, by the processor, a respective weight based on historical performance of the segment in predicting a CNV at the locus of interest, and generating, by the processor, a weighted score based on the respective weight and the respective segmented copy number score. The method may also include applying, by the processor, a probabilistic machine-learning (ML) copy number classifier to generate a probability of the CNV at the locus of interest based on an aggregate of the weighted score of each segment.

In some examples, the method may further include training the probabilistic ML copy number classifier based on a maximum likelihood estimation.

In some examples, the method may further include training the probabilistic ML copy number classifier based on a Hidden Markov Model.

In some examples, the method may further include training the probabilistic ML copy number classifier based on Bayesian inference.

In some examples, the disclosure relates to a method, implemented by a processor, of determining a genetic state of a locus of interest of a genetic material in a sample of a subject. The method may include detecting, by the processor, a genetic state associated with a segment in the genetic material in the sample, generating, by the processor, a probability that the locus of interest outside the segment is also in the genetic state, and predicting, by the processor, a disease condition of the subject based on the generated probability.

In some examples, the genetic material comprises cell free DNA derived from the locus of interest and wherein the genetic state comprises a copy number variant at the segment of the chromosome.

In some examples, generating the probability may include determining a level of deletion of the chromosome based on the copy number variant at the segment, wherein the generated probability rises as a proportional function with the level of deletion.

In some examples, generating the probability may include determining a physical distance between the segment and the locus of interest, wherein the generated probability rises as an inversely proportional function with the physical distance.

In some examples, detecting the genetic state may include accessing sequence reads generated from the sample, and analyzing sequence coverage information of the sequence reads.

In some examples, generating the probability may include generating the probability without sequence reads that cover the locus of interest.

In some examples, generating the probability may include generating the probability without genotyping data for the locus of interest.

In some examples, the locus of interest is associated with the disease condition.

In some examples, the locus of interest comprises a Human Leukocyte Antigens (HLA) locus.

In some examples, one or more target genes in the locus of interest and one or more reference genes in the segment are not within the same gene regulation pathway.

In some examples, one or more target genes in the locus of interest are in genetic linkage with one or more reference genes in the segment. In these examples, the method may further include identifying one or more segments, including the segment, to analyze for detection of copy number variants based on the genetic linkage.

In some examples, the genetic state includes at least one of: a copy number variation, an aneuploidy, a segmental loss, a fusion, or an indel.

In some examples, the method may further include determining a treatment based on the predicted disease condition.

In some examples, detecting the genetic state includes determining that the segment includes a copy number loss based on probe data for probes directed to the segment. In these examples, generating a probability that the locus of interest outside the segment is also in the genetic state includes determining that the locus of interest also includes a copy number loss.

In some examples, the disclosure relates to a method, implemented by a processor, of predicting a disease state of an individual based on a predicted state of a locus of interest of genetic material of the individual. The method may include accessing, by the processor, probe data for a plurality of segments in the genetic material, the plurality of segments being distinct from the locus of interest, determining, by the processor, whether or not each segment of the plurality of segments has a respective copy number loss based on the probe data, generating, by the processor, without probe data of probes directed to the locus of interest, a probability that the locus of interest also includes a copy number loss based on the determination of whether or not each segment of the plurality of segments has a respective copy number loss, and predicting, by the processor, a disease condition of the subject based on the determined probability.

In some examples, the disclosure relates to a method, implemented by a processor. The method may include accessing, by the processor, probe data for probes directed to a segment, determining, by the processor, that a segment includes a first copy number loss mutation based on probe data for probes directed to the segment, generating, by the processor, a probability that a locus of interest outside the segment includes a second copy number loss based on the determination that the segment includes the first copy number loss, and predicting, by the processor, a disease condition of the subject based on the determined probability.

In some examples, the disclosure relates to a method of inhibiting Poly Adenosine diphosphate-Ribose Polymerase (PARP) to treat cancer. The method may be implemented by a processor and may include detecting a genetic state associated with a segment in genetic material of a sample of a subject, generating a probability that a locus of interest outside the segment is also in the genetic state, the genetic state of the locus of interest being associated with cancer, and administering, based on the probability, a therapeutic agent to inhibit an ability of cancer cells to repair nucleic acid. In some examples, administering the therapeutic agent comprises administering a PARP inhibitor.

In some examples, the disclosure relates to a method of enhancing immune response to cancer cells. The method may be implemented by a processor and may include detecting a genetic state associated with a segment in genetic material of a sample of a subject, generating a probability that a locus of interest outside the segment is also in the genetic state, the genetic state of the locus of interest being associated with an ability of cancer cells to evade immune response, and administering, based on the probability, a therapeutic agent to block inhibition of the immune response to thereby enhance the immune response to the cancer cells. In some examples, administering the therapeutic agent comprises administering a Programmed cell Death (PD)-1 inhibitor. In some examples, administering the therapeutic agent comprises administering a Programmed cell Death (PD)-L1 inhibitor.

It should be noted that direct measurement of the locus of interest may augment, or improve probabilistic determinations described herein. In other words, the existence of direct measurements of a locus of interest may augment, not preclude, the systems and methods disclosed herein. Furthermore, although examples described herein may include the HLA locus, other loci of interest may be modeled as well such as the BRCA1/BRCA2 loci.

In some embodiments, the results of the systems and methods disclosed herein are used as an input to generate a report. The report may be in a paper or electronic format. For example, the classification of whether or not a locus of interest has a copy number variation, as determined by the methods and systems disclosed herein, can be displayed directly in such a report. Alternatively or additionally, the report may contain information relating to information derived from the classification of whether or not a locus of interest has a copy number variation, such as a diagnosis, a prognosis or a therapeutic recommendation.

The various steps of the methods disclosed herein, or steps carried out by the systems disclosed herein, may be carried out at the same or different times, in the same or different geographical locations, e.g. countries, and/or by the same or different people.

### BRIEF DESCRIPTION OF THE FIGURES

Features of the present disclosure may be illustrated by way of example and not limited in the following figure(s), in which like numerals indicate like elements, in which:

FIG. 1 illustrates an example of a system for identifying copy number variants in a sample of a subject, according to an implementation of the disclosure.

FIG. 2 illustrates a schematic diagram of an example of generating CNV calls for a sequence of interest that includes a locus of interest, according to an implementation of the disclosure.

FIG. 3 illustrates a schematic diagram of an example of identifying segments of a sequence of interest for determining copy number variation at a locus of interest, according to an implementation of the disclosure.

FIGS. 4A-C each illustrate a respective schematic diagram of segmented copy number scores based on the copy number score illustrated in FIG. 2 and the segments illustrated in FIG. 3, according to an implementation of the disclosure.

FIG. 5 illustrates a block diagram of an example of modeling the predictiveness of segments in determining CNV at a locus of interest, according to an implementation.

FIG. 6 illustrates a method of identifying CNV at a locus of interest in a sample of a subject, according to an implementation of the disclosure.

FIG. 7 illustrates a method of identifying a genetic state at a locus of interest in a sample of a subject, according to an implementation of the disclosure.

FIG. 8 illustrates a method of identifying CNL at a locus of interest in a sample of a subject, according to an implementation of the disclosure.

### DEFINITIONS

A direct measurement of a locus of interest may refer to data obtained by sequencing, probing (through hybridization probes), or otherwise conducting a biological experiment on the underlying DNA, RNA, methylation, histone marks or other genetic material at the locus of interest to ascertain the genetic or epigenetic state of the locus of interest and analyzing the obtained data. Thus, a direct measurement may involve both obtaining the data and analyzing the data. For example, a direct measurement may involve obtaining one or more sequence reads for a locus of interest and generating an alignment of the one or more sequence reads against a reference sequence, where such alignment is above a quality threshold for analysis. For example, a direct measurement of a locus of interest for copy number analysis may include obtaining sequence reads that at least partially cover the locus of interest and analyzing coverage depth based on the sequence reads that align to a reference genome above a quality threshold. The quality threshold may vary depending on the particular locus of interest involved. Examples of quality thresholds include a minimum nucleotide overlap and/or minimum alignment identity or similarity. The minimum nucleotide overlap may include, without limitation, a minimum overlap of at least about 1 base, 2 bases, 4 bases, 4 bases, 5 bases, 10 bases, 15 bases, 40 bases, 25 bases, 40 bases, 45 bases, 40 bases, 45 bases, 50 bases, 55 bases, 60 bases, 65 bases, 70 bases, 75 bases, 80 bases, 85 bases, 90 bases, 95 bases, or 100 bases. The minimum alignment identity or similarity may be at least about 5%, 10%, 15%, 40%, 25%, 40%, 45%, 40%, 45%, 50%, 55%, 60%, 65%, 70%, 75%, 80%, 85%, 90%, 95%, or more. It should be noted that data may be unavailable for analysis because the data was not obtained, such as because of difficulty in obtaining sequence reads, and/or the data was obtained but is difficult to analyze because the quality threshold has not been met.

A subject refers to an animal, such as a mammalian species (preferably human) or avian (e.g., bird) species, or other organism, such as a plant. More specifically, a subject can be a vertebrate, e.g., a mammal such as a mouse, a primate, a simian or a human. Animals include farm animals, sport animals, and pets. A subject can be a healthy individual, an individual that has symptoms or signs or is suspected of having a disease or a predisposition to the disease, or an individual that is in need of therapy or suspected of needing therapy.

A genetic variant refers to an alteration, variant or polymorphism in a nucleic acid sample or genome of a subject. Such alteration, variant or polymorphism can be with respect to a reference genome, which may be a reference genome of the species (e.g., for human, hG19 or hG38), the subject or other individual. Variations include one or more single nucleotide variations (SNVs), insertions, deletions, repeats, small insertions, small deletions, small repeats, structural variant junctions, variable length tandem repeats, and/or flanking sequences, CNVs, transversions, gene fusions and other rearrangements are also forms of genetic variation. A variation can be a base change, insertion, deletion, repeat, copy number variation, transversion, or a combination thereof.

A cancer marker is a genetic variant associated with presence or risk of developing a cancer. A cancer marker can provide an indication a subject has cancer or a higher risk of developing cancer than an age and gender matched subject of the same species that does not have the cancer marker. A cancer marker may or may not be causative of cancer.

A barcode is a short nucleic acid (e.g., less than 500, 100, 50 or 10 nucleotides long), used to label nucleic acid molecules to distinguish nucleic acids from different samples (e.g., representing a sample index), or different nucleic acid molecules in the same sample (e.g., representing a molecular barcode), of different types, or which have undergone different processing. Barcodes can be single stranded, double-stranded or at least partially double-stranded. Tags can have the same length or varied lengths. Tags can be blunt-end or have an overhang. Tags can be attached to one end or both ends of the nucleic acids. Barcodes can be decoded to reveal information such as the sample of origin, form or processing of a nucleic acid. Tags can be used to allow pooling and parallel processing of multiple samples comprising nucleic acids bearing different barcodes and/or sample indexes with the nucleic acids subsequently being deconvoluted by reading the barcodes. Additionally, or alternatively, barcodes can be used to distinguish different molecules in the same sample. This includes uniquely barcoding each different molecule in the sample, or non-uniquely barcoding each molecule. In the case of non-unique barcoding, barcodes with a limited number of different sequences may be used such that different molecules can be distinguished based on their start/stop position where they map on a reference genome in combination with at least one barcode. Typically then, a sufficient number of different barcodes are used such that there is a low probability (e.g. <10%, <5%, <1%, or <0.1%) that any two molecules having the same start/stop also have the same barcode. Some barcodes include multiple molecular identifiers to label samples, forms of molecule within a sample, and molecules within a form having the same start and stop points. Such barcodes can exist in the form Ali, wherein the letter indicates a sample type, the Arabic number indicates a form of molecule within a sample, and the Roman numeral indicates a molecule within a form.

Adapters are short nucleic acids (e.g., less than 500, 100 or 50 nucleotides long) usually at least partly double-stranded for linkage to either or both ends of a sample nucleic acid molecule. Adapters can include primer binding sites to permit amplification of a nucleic acid molecule flanked by adapters at both ends, and/or a sequencing primer binding site, including primer binding sites for next generation sequencing (NGS). Adapters can also include binding sites for capture probes, such as an oligonucleotide attached to a flow cell support. Adapters can also include a barcode as described above. Barcodes are preferably positioned relative to primer and sequencing primer binding sites, such that a barcode is included in amplicons and sequence reads of a nucleic acid molecule. Adapters of the same or different sequence can be linked to the respective ends of a nucleic acid molecule. Sometimes an adapter of the same sequence is linked to the respective ends except that the barcode is different. A preferred adapter is a Y-shaped adapter in which one end is blunt ended or tailed as described herein, for joining to a nucleic acid molecule, which is also blunt ended or tailed with one or more complementary nucleotides. Another preferred adapter is a bell-shaped adapter, likewise with a blunt or tailed end for joining to a nucleic acid to be analyzed.

As used herein, the term “sequencing” refers to any of a number of technologies used to determine the sequence of a biomolecule, e.g., a nucleic acid such as DNA or RNA. Exemplary sequencing methods include, but are not limited to, targeted sequencing, single molecule real-time sequencing, exon sequencing, electron microscopy-based sequencing, panel sequencing, transistor-mediated sequencing, direct sequencing, random shotgun sequencing, Sanger dideoxy termination sequencing, whole-genome sequencing, sequencing by hybridization, pyrosequencing, capillary electrophoresis, duplex sequencing, cycle sequencing, single-base extension sequencing, solid-phase sequencing, high-throughput sequencing, massively parallel signature sequencing, emulsion PCR, co-amplification at lower denaturation temperature-PCR (COLD-PCR), multiplex PCR, sequencing by reversible dye terminator, paired-end sequencing, near-term sequencing, exonuclease sequencing, sequencing by ligation, short-read sequencing, single-molecule sequencing, sequencing-by-synthesis, real-time sequencing, reverse-terminator sequencing, nanopore sequencing, 454 sequencing, Solexa Genome Analyzer sequencing, SOLiD™ sequencing, MS-PET sequencing, and a combination thereof. In some implementations, sequencing can be performed by a gene analyzer such as, for example, gene analyzers commercially available from Illumina or Applied Biosystems.

The phrase “next generation sequencing” or NGS refers to sequencing technologies having increased throughput as compared to traditional Sanger- and capillary electrophoresis-based approaches, for example, with the ability to generate hundreds of thousands of relatively small sequence reads at a time. Some examples of next generation sequencing techniques include, but are not limited to, sequencing by synthesis, sequencing by ligation, and sequencing by hybridization.

The phrase “sequencing run” refers to any step or portion of a sequencing experiment performed to determine some information relating to at least one biomolecule (e.g., a nucleic acid molecule such as DNA or RNA).

DNA (deoxyribonucleic acid) is a chain of nucleotides comprising four types of nucleotides; adenine (A), thymine (T), cytosine (C), and guanine (G). RNA (ribonucleic acid) is a chain of nucleotides comprising four types of nucleotides; A, uracil (U), G, and C. Certain pairs of nucleotides specifically bind to one another in a complementary fashion (called complementary base pairing). In DNA, adenine (A) pairs with thymine (T) and cytosine (C) pairs with guanine (G). In RNA, adenine (A) pairs with uracil (U) and cytosine (C) pairs with guanine (G). When a first nucleic acid strand binds to a second nucleic acid strand made up of nucleotides that are complementary to those in the first strand, the two strands bind to form a double strand. As used herein, “nucleic acid sequencing data,” “nucleic acid sequencing information,” “nucleic acid sequence,” “nucleotide sequence”, “genomic sequence,” “genetic sequence,” or “fragment sequence,” or “nucleic acid sequence read” denotes any information or data that is indicative of the order of the nucleotide bases (e.g., adenine, guanine, cytosine, and thymine or uracil) in a molecule (e.g., a whole genome, whole transcriptome, exome, oligonucleotide, polynucleotide, or fragment) of a nucleic acid such as DNA or RNA. It should be understood that the present teachings contemplate sequence information obtained using all available varieties of techniques, platforms or technologies, including, but not limited to: capillary electrophoresis, microarrays, ligation-based systems, polymerase-based systems, hybridization-based systems, direct or indirect nucleotide identification systems, pyrosequencing, ion- or pH-based detection systems, and electronic signature-based systems.

A “polynucleotide”, “nucleic acid”, “nucleic acid molecule”, or “oligonucleotide” refers to a linear polymer of nucleosides (including deoxyribonucleosides, ribonucleosides, or analogs thereof) joined by internucleosidic linkages. Typically, a polynucleotide comprises at least three nucleosides. Oligonucleotides often range in size from a few monomeric units, e.g. 3-4, to hundreds of monomeric units. Whenever a polynucleotide is represented by a sequence of letters, such as “ATGCCTG,” it will be understood that the nucleotides are in 5′→3′ order from left to right and that “A” denotes deoxyadenosine, “C” denotes deoxycytidine, “G” denotes deoxyguanosine, and “T” denotes thymidine, unless otherwise noted. The letters A, C, G, and T may be used to refer to the bases themselves, to nucleosides, or to nucleotides comprising the bases, as is standard in the art.

A reference sequence is a known sequence used for purposes of comparison with experimentally determined sequences. For example, a known sequence can be an entire genome, a chromosome, or any segment thereof. A reference typically includes at least 10¹, 10³, 10⁶, 10⁹ or more nucleotides. A reference sequence can align with a single contiguous sequence of a genome or chromosome or can include non-contiguous segments aligning with different regions of a genome or chromosome. Reference human genomes include, e.g., hG19 and hG38.

The term “designated position” in a reference sequence refers to a genomic coordinate in the reference sequence.

A first single stranded nucleic acid sequence overlaps with a second single stranded sequence if the first nucleic acid sequence or its complement and the second nucleic acid sequence or its complement align with overlapping but non-identical segments of a contiguous reference sequence, such as the sequence of a human chromosome. A fully or partially double-stranded nucleic acid overlaps with another fully or partially double-stranded nucleic acid if either of its strands overlaps those of the other nucleic acid.

A “C” to “T” variant or conversion refers to the presence of base “T” in a sequenced polynucleotide at a coordinate position occupied in a reference sequence by base “C”. A “G” to “A” variant or conversion refers to the presence of base “A” in a sequenced polynucleotide at a coordinate position occupied in a reference sequence by base “G”.

A nucleic acid molecule can be conceptually divided into a 5′ terminal end, an internal portion and a 3′ terminal end. Terminal ends can be designated based a predetermined number of nucleotides from the terminus. For example, the 5′ terminal end be represented by, e.g., the 20 terminal nucleotides to the 5′ end. The 3′ terminal end be represented by, e.g., the 20 terminal nucleotides to the 3′ end. Alternatively, the nucleic acid molecule can be divided into a terminal portion, as described, and a remainder.

Genetic linkage refers to the tendency of two or more nucleic acid sequences to be inherited together due to the absence of genetic recombination during meiosis. For example, a first locus and a second locus may be observed to be inherited together and may be determined to be genetically linked.

A physical distance may refer to a number of nucleotides between two nucleotide positions. For example, a physical distance between a first locus and a second locus may be one or more nucleotides that are in between the first locus and the second locus on a reference sequence.

The terms “processing”, “calculating”, and “comparing” can be used interchangeably. The term can refer to determining a difference, e.g., a difference in number or sequence. For example, gene expression, copy number variation (CNV), indel, and/or single nucleotide variant (SNV) values or sequences can be processed.

Adapters are an artificially synthesized sequence that can be coupled to a nucleic acid molecule or a polynucleotide sequence by any approach including ligation, hybridization, and/or amplification. Adapters are usually at least partly double-stranded for linkage to either or both ends of a sample nucleic acid molecule. Adapters can include primer binding sites to permit amplification of a nucleic acid molecule flanked by adapters at both ends, and/or a sequencing primer binding site, including primer binding sites for next generation sequencing (NGS). Adapters can also include binding sites for capture probes, such as an oligonucleotide attached to a flow cell support. Adapters can also include a barcode as described above. Tags are preferably position relative to primer and sequencing primer binding sites, such that a tag is included in amplicons and sequence reads of a nucleic acid molecule. The same or different adapters can be linked to the respective ends of a nucleic acid molecule. Sometimes the same adapter is linked to the respective ends except that the tag is different. A preferred adapter is a Y-shaped adapter in which one end is blunt ended or tailed as described herein, for joining to a nucleic acid molecule, which is also blunt ended or tailed with one or more complementary nucleotides. Another preferred adapter is a bell-shaped adapter, likewise with a blunt or tailed end for joining to a nucleic acid to be analyzed. Other adapters include hairpin adapters.

### DETAILED DESCRIPTION

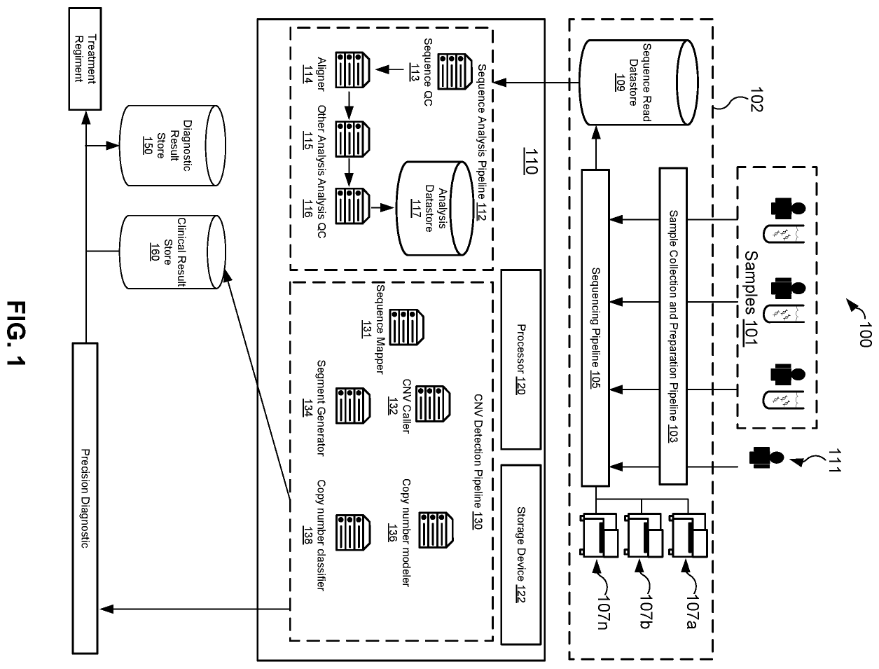

FIG. 1 illustrates an example of a system 100 for identifying copy number variants in a sample 101 of a subject 111, according to an implementation of the disclosure. The system 100 may process one or more samples 101 from the subject 111 to generate sequence reads for variant detection. The system 100 may include a laboratory system 102, a computer system 110, and/or other components. It should be noted that the laboratory system 102 and the computer system 110 may be remote from one another, and connected to one another through a computer network (not illustrated).

The laboratory system 102 may include a sample collection and preparation pipeline 103, a sequencing pipeline 105, a sequence read datastore 109, and/or other components. In some implementations, the sample collection and preparation pipeline 103 may be configured to reduce instances of laboratory-induced variants, which may lead to false positive reporting. For example, the sample collection and preparation pipeline 103 may be modified to increase by 10-fold (10×) or more a molar concentration of primers used for Polymerase Chain Reaction (PCR). Such 10-fold or more excess primer concentration may reduce instances of incomplete extension during PCR cycles by competitively binding available templates during PCR. As such, excess primer use may reduce instances of PCR chimer formation, which may result in laboratory-induced variants such as nucleic acid rearrangements. The sequencing pipeline 105 may include one or more sequencing devices 107 (illustrated in FIG. 1 as sequencing devices 107a . . . n).

The computer system 110 may include a sequence analysis pipeline 112, a processor 120, a storage device 122, a copy number variant (CNV) detection pipeline 130, and/or other components. The sequence analysis pipeline 112 may access data from the sequence read datastore 109 and process the data for analysis by the CNV detection pipeline 130. For example, the sequence analysis pipeline 112 may include a sequence quality control (QC) component 113, aligner 114, other analysis components 115, and an analysis QC component 116. Output from the sequence analysis pipeline 112 may be stored in an analysis datastore 117.

The sequence QC component 113 may perform quality control on the sequence read data from the sequencing pipeline 105. It should be noted that some or all of the operations of sequence QC component 113 may be performed at the laboratory system 102. The aligner 114 may align sequence reads using various sequence alignment techniques. Such alignment may be performed against a reference sequence. The analysis QC component 116 may perform quality control analysis on the analysis.

Generally speaking, the processor 120 may implement (be programmed by) various components of the CNV detection pipeline 130, such as a sequence read mapper 131, a CNV caller 132, a segment generator 134, a copy number modeler 136, a copy number classifier 138, and/or other components. Alternatively, it should be noted that each of these components of the CNV detection pipeline 130 may include a hardware module. Although illustrated separately for convenience, one or more of the various components or instructions, such as the segment generator 134, the copy number modeler 136, the copy number classifier 138, and/or other components may be integrated with one another. In any event, the CNV detection pipeline 130 may cause the computer system 110 to generate copy number variant predictions, such as copy number loss predictions, of a locus of interest, diseases from the variants (precision diagnostics), and/or treatment regiments. The precision diagnostic and treatment regimen may be stored in a repository such as clinical result store 160 or diagnostic result store 150.

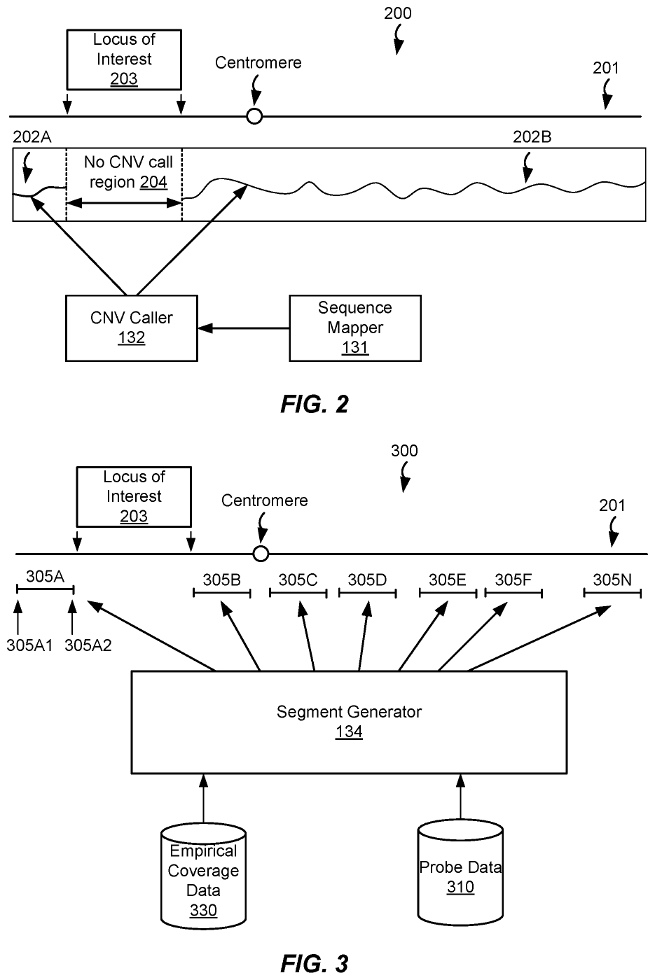

To describe examples of the operations of the CNV detection pipeline 130, reference will first be made to FIG. 2, which illustrates a schematic diagram of an example of generating CNV calls for a sequence of interest 201 that includes a locus of interest 203, according to an implementation of the disclosure. Although a centromere is shown in the illustrated example, such centromere is not necessarily required.

The sequence of interest 201 may include a chromosome or other nucleic acid sequence and the locus of interest 203 may include a location on the sequence of interest 201 for which CNV, such as CNL, is to be predicted. In some examples, the locus of interest 203 may be associated with a start position and an end position (indicated by downward arrows) on the sequence of interest 201. In a particular example, the sequence of interest 201 may include human chromosome 6 and the locus of interest 203 may include a locus that encodes or is otherwise associated with encoding an HLA protein.

To make CNV calls, the sequence read mapper 131 may align the sequence reads to a reference sequence. The reference sequence may include a sequence, such as a whole genome sequence, of a species of the subject 111. For example, if the subject 111 is human, the reference sequence may be the hG19 or hG38 whole genome sequence. In some examples, the reference sequence may be truncated to include only the sequence of interest 201. For example, the hG19 or hG38 whole genome sequence may be truncated to include only the sequence corresponding to chromosome 6.

In some examples, the CNV caller 132 may generate CNV calls based on sequence read coverage derived from the alignment generated by the sequence read mapper 131. The CNV caller 132 may do so based on a baseline expectation of sequence read coverage when no CNV is present. In other words, if the sequence read coverage in a region deviates from the baseline expectation for that region, the CNV caller 132 may determine that CNV has occurred in that region. In some examples, the deviation may be higher than the baseline expectation, indicating a duplication may have occurred in the region. In other examples, the deviation may be lower than the baseline expectation, indicating a CNL may have occurred in the region. An example of a method for generating a copy number variation call is described in PCT application no. PCT/US2020/020174, filed Feb. 27, 2020, which is incorporated by reference in its entirety herein.

In some examples, the CNV caller 132 may generate a copy number score 202 (illustrated as copy number score 202A, 202B) that indicates the CNV calls across the sequence of interest 201. For example, the copy number score 202 may represent copy number determinations of different regions along the sequence of interest 201. Each of such different regions may be predicted by the CNV caller 132 to have or not have CNV at the region.

In some examples, the locus of interest 203 may include features such as genetic polymorphism, extended sequence nucleotide repeats, or low sequence complexity that render it difficult to make direct measurements of the locus of interest 203. The HLA locus will be described as an example of a locus of interest 203 for which direct measurements are unavailable because data covering the HLA locus has not been obtained and/or analysis is difficult due to the features of the HLA locus.

To illustrate, an HLA locus encoding an HLA protein may be polymorphic due to its role in immune response. Such polymorphic variability is believed to facilitate recognition of a wide range of antigens to mediate an immune response to different pathogens (or cancer cells for tumor suppression). Other types of biological features, such as repetitive nucleic acid sequences, may also render it difficult to make direct CNV calls due to limitations of laboratory techniques and/or computational limitations of being unable to disambiguate such biological features. As such, even if sequence reads that cover the locus of interest 203 are obtained, such sequence reads may be unavailable for analysis because the CNV caller 132 may be unable to accurately align the sequence reads for accurate CNV calls. Thus, any sequence reads that cover the HLA locus may be unavailable for analysis. In this example, the copy number score 202 may have a “no CNV call region” 204 at the locus of interest 203.

Some regions of the sequence of interest 201 may be determined to correlate with the copy number of the locus of interest 203. For example, these regions may be physically linked with the locus of interest 203. In a particular example, CNL at a first region of the sequence of interest 201 may observed to co-occur with CNL at the locus of interest 203 according to empirical data. This may be due to genetic linkage between the first region and the locus of interest 203 such that a loss (or other variant) in one may cause or be otherwise be associated with a loss in the other. Such linkage may be separate from being linked by virtue of a genetic pathway in which two loci are involved in the same genetic pathway, such as where one locus is a regulator of another locus or other genetic pathway linkage.

To leverage the correlation of some regions of the sequence of interest 201 with the locus of interest, segment generator 124 may analyze such correlations to generate segments 305 of the sequence of interest 201 that correspond to such regions. For example, FIG. 3 illustrates a schematic diagram 300 of an example of identifying segments 305 (illustrated as segments 305A . . . N) of a sequence of interest 201 for determining copy number variation at a locus of interest 203, according to an implementation of the disclosure. A “segment” 305 may refer to a region of a sequence of interest 201 whose copy number is correlated with the copy number of the locus of interest 203. Thus, the copy number of a segment 305 may be predictive for the copy number of the locus of interest 203. The segment generator 134 may identify the segments 305 based on probe data 310, empirical coverage data 330, and/or other sources. The region of the sequence of interest 201 may be associated with a start position 305A1 and an end position 305A2. Other segments 305B-N may each also be associated with a start and end position (not illustrated for convenience).

In some examples, the segment generator 134 may identify the segments 305 at the probe-level based on the probe data 310. The probe data 310 may include data relating to one or more probes that hybridize to a region of the sequence of interest 201 (such region may be referred to as a hybridization region). The hybridization may be detectable through various techniques. In some instances, such hybridization may be quantified to determine a copy number at the hybridization region. For example, based on a strength of fluorescence of a hybridized probe, the copy number of the hybridization region may be determined. Thus, the copy number of hybridization regions across the sequence of interest 201 may be quantifiable. In some examples, the segment generator 134 may use hybridization regions to define the segments 305. For example, the segment generator 134 may use the start and end position of a hybridization region as a start and end position of a segment 305.

Alternatively, or additionally, the segment generator 134 may learn to identify segments 305 based on empirical coverage data 330. The empirical coverage data 330 may include sequence read coverage data across sequence of interest 201. In these examples, the segments 305 may be selected by sampling windows along the sequence of interest 201. A window as used herein may refer to a start and stop position on the sequence of interest 201 in which copy number may be predicted based on the underlying sequence read coverage. For example, the CNV caller 132 may be used to generate copy number predictions across the sequence of interest 201. Each window may represent a copy number prediction at a given start and stop position of the sequence of interest 201. The segment generator 134 may randomly select windows or may traverse the sequence of interest 201 in a sliding window fashion in which a preset window size is selected and slid across in increments of one or more nucleotides. Once selected (randomly, sliding window, or otherwise), each window may be assessed to determine whether a copy number score in that window is correlated with copy number of the locus of interest 203.

It should be noted that the identified segments 305 may be stored in a data store for later access. In any event, once the segments 305 have been identified and/or accessed from the data store, the segment generator 134 may use the segments 305 to identify CNV calls from the CNV caller 132 that may correlate with CNV at the locus of interest 203.

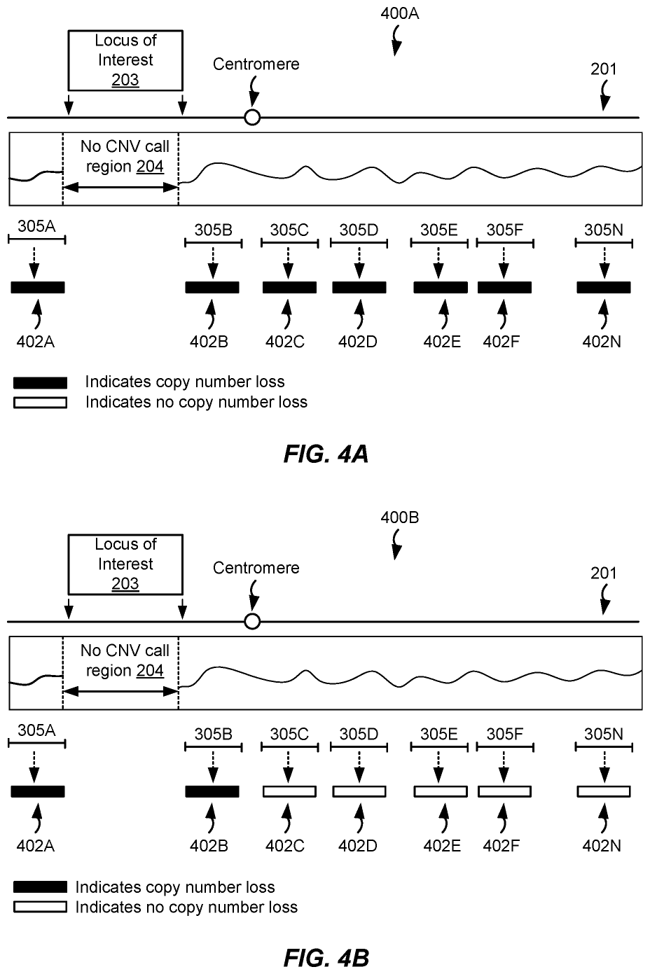

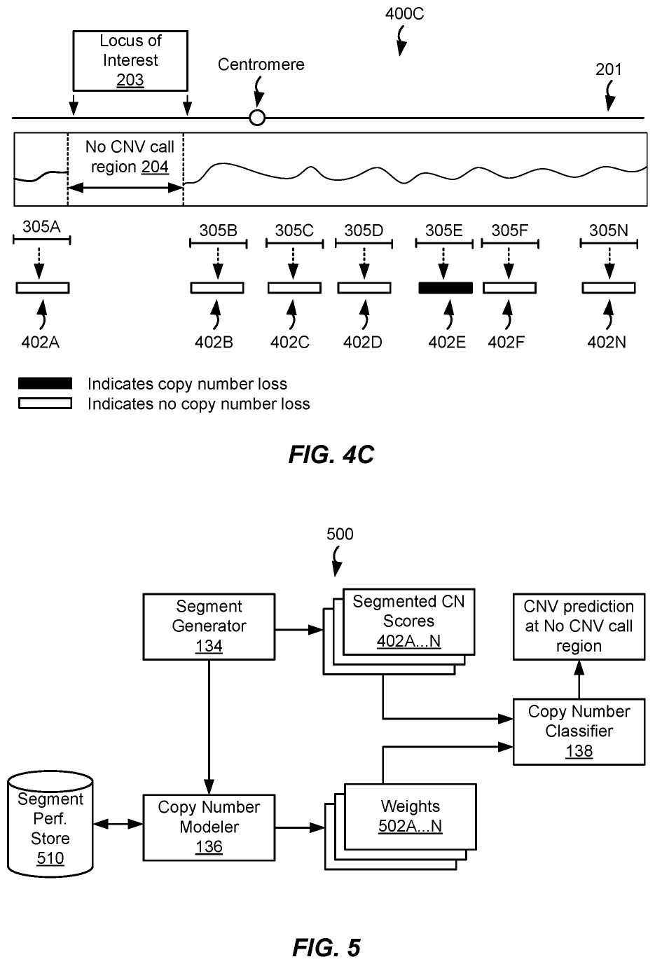

For example, FIGS. 4A-C each illustrate a respective schematic diagram 400A-C of segmented copy number scores 402 (illustrated as segmented copy number scores 402A-N) based on the copy number score 202 illustrated in FIG. 2 and the segments 305 illustrated in FIG. 3, according to an implementation of the disclosure. A “segmented copy number score” 402 may refer to a value that represents a determination of a copy number at a corresponding segment 305. For example, the segmented copy number score 402A may represent a CNL at segment 305A of the sequence of interest 201. It should be noted that various examples that follow may refer to sequence coverage-based copy number determinations of segments 305. However, probe-based measurements may be used instead or in addition to these examples. For instance, a given segment 305 may correspond to a hybridization region at which a probe hybridizes to the sequence of interest 201. In this example, the segmented corresponding segmented copy number score 402 may reflect a level of fluorescence or other hybridization measurement that may be used to determine CNV at the hybridization region.

Such prediction may be based on the CNV caller 132 at a region of the sequence of interest 201 corresponding to the segment 305. For example, the segment generator 134 may generate a segmented copy number score 402A for segment 305A, a segmented copy number score 402B for segment 305B, and so forth. The segmented copy number score 402A may refer to a prediction from the CNV caller 132 at the region corresponding to segment 305A.

Referring to FIG. 4A, all of the illustrated segmented copy number scores 402 may indicate CNL at respective segments 305. The foregoing may indicate CNL also at the locus of interest 203. Referring to FIG. 4B, segmented copy number scores 402A and 402B may both indicate CNL at respective segments 305A and 305B. Segmented copy number scores 402C-402N may each indicate no CNL at respective segments 305C-N. However, because the segments 305A and 305B are both on the same arm of the chromosome (same side of the centromere in the illustrated example). Thus, the foregoing may also indicate CNL at the locus of interest 203, albeit with less probability than the example illustrated in FIG. 4A. As illustrated in FIG. 4B, the location of the segments 305 (in this case whether on the same arm as the locus of interest 203) may be a factor in predicting CNL. Referring to FIG. 4C, only the segmented copy number scores 402E indicates CNL at segment 305E. Thus, as compared to the examples illustrated in FIGS. 4A and 4B, the example illustrated in FIG. 4C may result in the lowest probability of CNL at the locus of interest 203.

As illustrated in FIGS. 4A-C, the segmented copy number scores 402, including the location of respective segments 305 relative to the locus of interest 203, may impact a given segment's 305 predictiveness of CNV at the locus of interest 203. Put another way, various factors may impact whether or not CNV at a segment 305 is predictive of CNV at the locus of interest 203. Such factors may include, without limitation, genetic linkage, distance between the locus of interest 203 and the segment 305, size of the segment 305 (for example, copy number loss spanning a larger area may indicate loss of the locus of interest 203), and/or other factors. In some examples, because of the variable nature in which a given segment 305 may or may not be predictive of a locus of interest 203, the segments 305 may be analyzed to determine predictiveness based on empirical data of prior performance of the segments 305.

For example, FIG. 5 illustrates a block diagram of an example of modeling the predictiveness of segments 305 in determining CNV at a locus of interest 203, according to an implementation. The copy number modeler 136 may learn from the historical performance of a given segment 305 at predicting CNV at the locus of interest 203. For example, the segment performance store 510 (illustrated as segment perf. store 510) may store performance data relating to segments 305 across multiple samples 101 of multiple subjects 111. Each segment 305 stored in the segment perf. store 510 may relate to a segmented copy number score 402 derived from a particular sample 101 of a particular subject 111. The segment perf. store 510 may store the segmented copy number score 402 for the segment 305 in association with empirical data indicating whether there was CNV at the locus of interest 203 in the sample 101. The copy number modeler 136 may access the segment perf. store 510 to access performance data relating to each segment 305. Thus, for each segment 305, the copy number modeler 136 may access historical performance data.

In some examples, based on learning from the historical performance of segments 305 across different samples 101 of different subjects 111, the copy number modeler 136 may assign a weight 502 (illustrated as weights 502A . . . N) for each segment 305. A weight 502 may refer to quantitative level of correlation between CNV (or no CNV) at each segment 305 and CNV (or no CNV) at the locus of interest 203. For example, a higher level of correlation between a given segment 305 and the locus of interest 203 means that the segmented copy number score 402 (the prediction of copy number associated with the given segment 305) is more likely than a lower level of correlation to be predictive of the copy number at the locus of interest 203. Put another way, a higher weight 502 for the given segment 305 means that there is a greater chance that if the given segment 305 exhibits CNL or other CNV then the locus of interest 203 will also exhibit the CNL or other CNV. A weight 502 may be assigned with a quantitative value, such as 0 to 1.0 for some neural networks, 0 to 100, and/or other quantitative value that may be used to adjust a level of predictiveness of a segment 305. In some examples, a given weight 502 may be initially assigned based on observations of historical performance by, for example, a researcher. In some of these examples, the weight 502 may then refined based on computational modeling.

To illustrate, suppose a segment 305C has no to low co-occurrence of calling a CNV at the segment 305 and observed CNV at the locus of interest 203 across different samples 101 and different subjects 111. On the other hand, a segment 305D may have high co-occurrence of calling a CNV at the segment 305 and observed CNV at the locus of interest 203 across different samples 101 and different subjects 111. The copy number modeler 136 may determine that the segment 305D is predictive of CNV at the locus of interest 203 but that the segment 305C is not predictive of CNV at the locus of interest 203. In this example, the copy number modeler 136 may assign a weight 502D for the segment 305D that is higher than a weight 502C for the segment 305C.

The copy number modeler 136 may therefore provide weights 502 that indicate predictiveness of corresponding segments 505 of the copy number of the locus of interest 203 based on a correlation of a corresponding segmented copy number score 402 with the copy number of the locus of interest 203.

In some examples, the copy number modeler 136 may do so based on a classification of the segment 305. Such classification may include a binary classification of whether the segment 305 is (or is not) predictive of the copy number of the locus of interest 203. In this sense, the copy number modeler 136 may include a binary classifier that generates a first binary probability that the segment 305 is predictive of the copy number of the locus of interest 203 and a second binary probability that the segment 305 is not predictive of the copy number of the locus of interest 203. For example, labeled data for training and validation may include a known CNV at the locus of interest 203. Features for such training and validation may include segmented copy number scores 402 and segments 305 associated with the known CNV at the locus of interest 203. The copy number modeler 136 may therefore be trained to identify segments 305 and/or combinations of segments 305 that are predictive of CNV at the locus of interest 203.

In some examples, the segmented copy number scores 402 and corresponding weights 502 may be used to generate respective weighted scores. For example, the segmented copy number score 402A (corresponding to segment 305A) may be weighted by the corresponding weight 502A. Likewise, the segmented copy number score 402B (corresponding to segment 305B) may be weighted by the corresponding weight 502B, and so forth. The copy number modeler 136, the copy number classifier 138, and/or other component may generate the weighted scores.

Regardless of which component generates the weighted scores, the copy number classifier 138 may aggregate the weighted scores to generate a classification of CNV at the locus of interest 203. For example, the classification of CNV at the locus of interest 203 may include a probability that there is CNV at the locus of interest 203 for the sample 101 of the subject 111. Such probability may be based on the weights learned from the populate of samples 101 of subjects 111 available for training the copy number modeler 136. If the probability is above a threshold probability, then CNL (or other CNV being assessed) at the locus of interest 203 of the sample 101 of the subject 111 may be determined. The threshold probability may be greater than 5%, greater than 10%, greater than 15%, greater than 25%, greater than 40%, greater than 45%, greater than 50%, greater than 55%, greater than 60%, greater than 65%, greater than 70%, greater than 75%, greater than 80%, greater than 85%, greater than 90%, greater than 95%, or more. In certain embodiments, the threshold probability is predetermined. In certain embodiments, the threshold probability is a combination of the probabilities across two or more loci of interest.

In some examples, the copy number classifier 138 may be a one-class classifier (OCC) that generates a single classification that a particular CNV exists at the locus of interest 203, a binary classifier that generates a binary classification of whether a particular CNV exists at the locus of interest 203, or a multi-class classifier that generates more than two classifications, where each of classification of the multi-class classification may correspond to a respective type of CNV at the locus of interest 203. In these examples, a first classification the multi-class classification may correspond to a first probability that a copy number loss exists at the locus of interest 203, a second probability that the duplication exists at the locus of interest 203, and/or other probabilities that other type of CNV exists at the locus of interest 203. Whether the copy number classifier 138 is an OCC, binary, or multi-class classifier may depend on the training and validation data used to train the copy number classifier 138 in these examples. In particular, for OCC classification, labeled data may correspond to a known type of CNV at the locus of interest 203. For binary classification two sets of labeled data may correspond to a known type of CNV at the locus of interest 203 and the known type of CNV not at the locus of interest 203. For multi-class classification, a plurality of sets of labeled data may correspond to a plurality of types of CNV known to be at the locus of interest 203.

In some examples, the copy number classifier 138 may be generated based on model parameters learned from historical data (such as from the segment perf. store 510) for ML models. In machine-learning, a model parameter may refer to a value that is fit to observed (training) data while minimizing error between the observed data and observed outcome. Thus, a model parameter may be used to generate a prediction on new data based on learned information from the observed data. Examples of model parameters may include network weights used in an artificial neural network, support vectors in a support vector machine, and coefficients in a linear regression or logistic regression.

In some examples, the model parameters for the copy number classifier 138 may be learned using probabilistic ML estimation, which may include a maximum likelihood estimation, Hidden Markov Model (HMM), Bayesian inference, and/or other techniques for estimating model parameters based on probabilistic ML. In these examples, the copy number classifier 138 may be referred to as a probabilistic ML copy number classifier.

In maximum likelihood estimation, values for parameters of a model may be determined such that the parameter values maximize the probability that the process described by the model produced the data that were actually observed. For example, if all observed regions show copy number loss. Various types of parameters may include a physical distance between a segment 305 and the most likely explanation is that the whole locus of interest 305, a nucleotide length of the segment 305, whether the segment 305 and the locus of interest 305 are on the same side of any centromere (or whether they are on the same chromosome arm, for example), and/or other variable that may impact the predictiveness of a given segment 305 in relation to a locus of interest 203.

HMMs are probabilistic models that generate a probability that hidden variables have a given value (such as CNV present or CNV not present at a locus of interest 203) based on observed variables (such as segmented copy number scores 402 for segments 305). For example, observed regions preceding and following the unobserved region would be used to infer the copy number state of the unobserved region.

In Bayesian modeling, a probability may be deduced from known probabilities. For example, the historical performance of a given segment 305 in predicting CNV at a locus of interest 203 may reflect a known probability from which a probability that there is a CNV at the locus of interest 203 may be deduced. In such an implementation, the copy number state of observed regions is used to infer the state at the region of interest.

It should be noted that although described herein as a way to generate a CNV call in the absence of a direct CNV call at the no CNV call region 204 (illustrated in FIG. 2), the systems and methods described herein may be used to supplement instances in which CNV calls are available as well. For example, data from the CNV caller 132 may be used in combination with the predicted CNV determination described herein, which may improve confidence of CNV calls at a locus of interest 203.

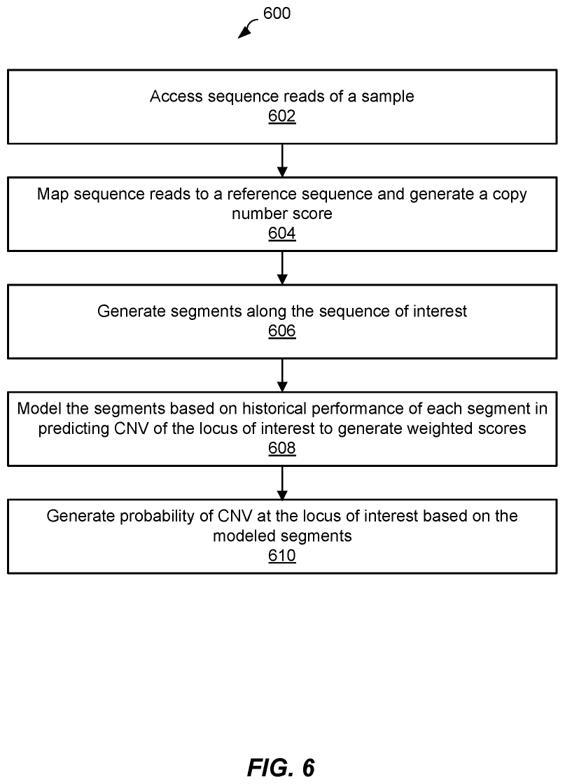

FIG. 6 illustrates a method 600 of identifying CNV at a locus of interest 203 in a sample 101 of a subject 111, according to an implementation of the disclosure. At 602, the method 600 may include accessing sequence reads of the sample 101 of the subject 111. For example, the sequence reads may be accessed from the sequence read datastore 109.

At 604, the method 600 may include mapping the sequence reads to a reference sequence and generating copy number scores based on the mapping. The reference sequence may include a sequence of interest (such as the sequence of interest 201). For example, the sequence read mapper 131 may align the sequence reads from the sample 101 of the subject 111 to the reference sequence. Based on the mapping, the CNV caller 132 may generate the copy number scores, which may indicate a copy number at a given region of the sequence of interest.

At 606, the method 600 may include generating segments (such as segments 305) along the sequence of interest. For example, the segment generator 134 may generate segments of the sequence of interest at the probe-level (using probe data 310), empirical coverage data (such as empirical coverage data 330), and/or other information. It should be noted that segments 305 generated based on probe data 310 may include an error window of M nucleotides upstream of a location of hybridization of the probe and/or N nucleotides downstream of the location of hybridization of the probe, where M and N represent integers that may or may not be equal to one another. In this manner the location of the segment 305 may be able to tolerate errors in alignment or underlying sequence for the probe data 310.

At 608, the method 600 may include modeling the segments based on historical performance of each segment in predicting CNV of the locus of interest to generate weights (such as weights 502) for each segment. For example, the copy number modeler 136 may generate the weights based on historical data from the segment perf. store 510. The copy number scores generated at 604 may each be weighted by a respective weight to generate weighted copy number scores.

At 610, the method 600 may include predicting copy number variant at the locus of interest based on the weighted copy number scores. For example, the copy number classifier 138 may determine a CNV prediction at the locus of interest in the sample 101 of the subject 111.

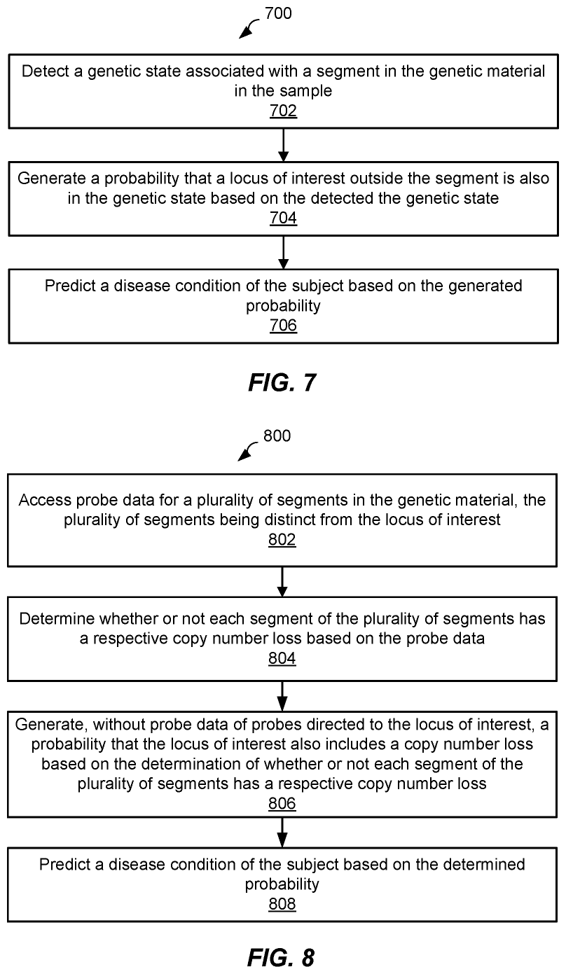

FIG. 7 illustrates a method 700 of identifying a genetic state at a locus of interest 203 in a sample 101 of a subject 111, according to an implementation of the disclosure.

At 702, the method 700 may include detecting a genetic state associated with a segment (such as a segment 305) in the genetic material in the sample (such as sample 101 of a subject 111). The genetic state may include a CNV (such as CNL), an aneuploidy, a normal (such as wildtype) state, and/or other state associated with the segment. The genetic material may include nucleic acids such as some or all of a genome, some or all of a chromosome, and/or other genetic material.

At 704, the method 700 may include generating a probability that a locus of interest (such as a locus of interest 203) outside the segment is also in the genetic state. The probability may be generated based on operations of the CNV caller 132, the segment generator 134, the copy number modeler 136, the copy number classifier 138, and/or other components of the computer system 110 illustrated in FIG. 1.

At 706, the method 700 may include predicting a disease condition of the subject based on the generated probability. For example, the disease condition may include cancer or other disease indicated by the detected genetic state of the locus of interest.

FIG. 8 illustrates a method of identifying CNL at a locus of interest 203 in a sample 101 of a subject 111, according to an implementation of the disclosure.

At 802, the method 800 may include accessing probe data for a plurality of segments (such as segments 305) in the genetic material, the plurality of segments being distinct from the locus of interest (such as locus of interest 203). In some examples, the probe data may include information indicating a copy number at a region in which a probe hybridizes to the genetic material. In these examples, each probe for which probe data is available may correspond to a segment. The genetic material may include nucleic acids such as some or all of a genome, some or all of a chromosome, and/or other genetic material.

At 804, the method 800 may include determining whether or not each segment of the plurality of segments has a respective copy number loss based on the probe data. At 806, the method 800 may include generating, without probe data of probes directed to the locus of interest, a probability that the locus of interest also includes a copy number loss based on the determination of whether or not each segment of the plurality of segments has a respective copy number loss.

At 808, the method 800 may include predicting a disease condition of the subject based on the determined probability. The probability may be generated based on operations of the CNV caller 132, the segment generator 134, the copy number modeler 136, the copy number classifier 138, and/or other components of the computer system 110 illustrated in FIG. 1.

The various methods 600-800 respectively depicted in FIGS. 6-8 may be accomplished using some or all of the system components described in detail above and, in some implementations, various operations may be performed in different sequences and various operations may be omitted. Additional operations may be performed along with some or all of the operations shown in the depicted flow diagrams. One or more operations may be performed simultaneously. Accordingly, the operations as illustrated (and described in greater detail below) are provided as example and, as such, should not be viewed as necessarily limiting.

Computer Implementation

The present methods can be computer-implemented, such that any or all of the steps described in the specification or appended claims other than wet chemistry steps can be performed in a suitable programmed computer. The computer can be a mainframe, personal computer, tablet, smart phone, cloud, online data storage, remote data storage, or the like. The computer can be operated in one or more locations.

Various operations of the present methods can utilize information and/or programs and generate results that are stored on computer-readable media (e.g., hard drive, auxiliary memory, external memory, server; database, portable memory device (e.g., CD-R, DVD, ZIP disk, flash memory cards), and the like.

The present disclosure also includes an article of manufacture for analyzing a nucleic acid population that includes a machine-readable medium containing one or more programs which when executed implement the steps of the present methods.

The disclosure can be implemented in hardware and/or software. For example, different aspects of the disclosure can be implemented in either client-side logic or server-side logic. The disclosure or components thereof can be embodied in a fixed media program component containing logic instructions and/or data that when loaded into an appropriately configured computing device cause that device to perform according to the disclosure. A fixed media containing logic instructions can be delivered to a viewer on a fixed media for physically loading into a viewer's computer or a fixed media containing logic instructions may reside on a remote server that a viewer accesses through a communication medium to download a program component.

The present disclosure provides computer control systems that are programmed to implement methods of the disclosure. The processor 120 may include a single core or multi core processor, or a plurality of processors for parallel processing. The storage device 122 may include random-access memory, read-only memory, flash memory, a hard disk, and/or other type of storage. The computer system 110 may include a communication interface (e.g., network adapter) for communicating with one or more other systems, and peripheral devices, such as cache, other memory, data storage and/or electronic display adapters. The components of the computer system 110 may communicate with one another through an internal communication bus, such as a motherboard. The storage device 122 may be a data storage unit (or data repository) for storing data. The computer system 110 may be operatively coupled to a computer network (“network”) with the aid of the communication interface. The network may be the Internet, an internet and/or extranet, or an intranet and/or extranet that is in communication with the Internet. The network in some cases is a telecommunication and/or data network. The network may include a local area network. The network may include one or more computer servers, which can enable distributed computing, such as cloud computing. The network, in some cases with the aid of the computer system 110, may implement a peer-to-peer network, which may enable devices coupled to the computer system 110 to behave as a client or a server.

The processor 120 may execute a sequence of machine-readable instructions, which can be embodied in a program or software. The instructions may be stored in a memory location, such as the storage device 122. The instructions can be directed to the processor 120, which can subsequently program or otherwise configure the processor 120 to implement methods of the present disclosure. Examples of operations performed by the processor 120 may include fetch, decode, execute, and writeback.

The processor 120 may be part of a circuit, such as an integrated circuit. One or more other components of the system 100 may be included in the circuit. In some cases, the circuit may include an application specific integrated circuit (ASIC).

The storage device 122 may store files, such as drivers, libraries and saved programs. The storage device 122 can store user data, e.g., user preferences and user programs. The computer system 110 in some cases may include one or more additional data storage units that are external to the computer system 110, such as located on a remote server that is in communication with the computer system 110 through an intranet or the Internet.

The computer system 110 can communicate with one or more remote computer systems through the network. For instance, the computer system 110 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 110 via the network.

Methods as described herein can be implemented by way of machine (e.g., computer processor) executable code stored on an electronic storage location of the computer system 110, such as, for example, on the storage device 122. The machine executable or machine readable code can be provided in the form of software. During use, the code can be executed by the processor 905. In some cases, the code can be retrieved from a storage unit and stored on the storage device 122 for ready access by the processor 120.

The code may be pre-compiled and configured for use with a machine having a processer adapted to execute the code, or can be compiled during runtime. The code can be supplied in a programming language that can be selected to enable the code to execute in a precompiled or as-compiled fashion.

Aspects of the systems and methods provided herein, such as the computer system 110, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk.

“Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible, storage media, “media” may include other types of (intangible) media.

“Storage” media, terms such as computer or machine “readable medium” refer to any tangible (such as physical), non-transitory, medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 110 can include or be in communication with an electronic display 935 that comprises a user interface (UI) for providing, for example, a report. Examples of UI's include, without limitation, a graphical user interface (GUI) and web-based user interface.

Methods and systems of the present disclosure can be implemented by way of one or more algorithms. An algorithm can be implemented by way of software upon execution by the processor 120.

Sample Collection and Analysis Pipeline

A sample 101 may be any biological sample isolated from a subject. Samples can include body tissues, such as known or suspected solid tumors, whole blood, platelets, serum, plasma, stool, red blood cells, white blood cells or leucocytes, endothelial cells, tissue biopsies, cerebrospinal fluid synovial fluid, lymphatic fluid, ascites fluid, interstitial or extracellular fluid, the fluid in spaces between cells, including gingival crevicular fluid, bone marrow, pleural effusions, cerebrospinal fluid, saliva, mucous, sputum, semen, sweat, urine. Samples are preferably body fluids, particularly blood and fractions thereof, and urine. Such samples include nucleic acids shed from tumors. The nucleic acids can include DNA and RNA and can be in double- and/or single-stranded forms. A sample can be in the form originally isolated from a subject or can have been subjected to further processing to remove or add components, such as cells, enrich for one component relative to another, or convert one form of nucleic acid to another, such as RNA to DNA or single-stranded nucleic acids to double-stranded. Thus, for example, a body fluid for analysis is plasma or serum containing cell-free nucleic acids, e.g., cell-free DNA (cfDNA).

In certain implementations, the polynucleotides can be enriched prior to sequencing. Enrichment can be performed for specific genomic target sequences. In certain embodiments, the specific genomic target sequences do not include the locus of interest. For example, the specific genomic target sequences may not include any portion of the locus of interest. In certain other implementations, enrichment can be performed nonspecifically. In some implementations, targeted regions of interest may be enriched with capture probes (“baits”) selected for one or more bait set panels using a differential tiling and capture scheme. A differential tiling and capture scheme uses bait sets of different relative concentrations to differentially tile (e.g., at different “resolutions”) across genomic regions associated with baits, subject to a set of constraints (e.g., sequencer constraints such as sequencing load, utility of each bait, etc.), and capture them at a desired level for downstream sequencing. These targeted genomic regions of interest may include regions of a subject's genome or transcriptome. In some implementations, biotin-labeled beads with probes to one or more regions of interest can be used to capture target sequences, optionally followed by amplification of those regions, to enrich for the regions of interest.

Sequence capture typically involves the use of oligonucleotide probes that hybridize to the target sequence. A probe set strategy can involve tiling the probes across a region of interest. Such probes can be, e.g., about 60 to 130 bases long. The set can have a depth of about 2×, 3×, 4×, 5×, 6×, 8×, 9×, 10×, 15×, 30×, 50×, or more. The effectiveness of sequence capture depends, in part, on the length of the sequence in the target molecule that is complementary (or nearly complementary) to the sequence of the probe.

In some implementations, the methods of the disclosure comprise selectively enriching regions from the subject's genome or transcriptome prior to sequencing. In other implementations, the methods of the disclosure comprise non-selectively enriching regions from the subject's genome or transcriptome prior to sequencing.

In certain implementations, sample index sequences are introduced to the polynucleotides after enrichment. The sample index sequences may be introduced through PCR or ligated to the polynucleotides, optionally as part of adapters.

The volume of plasma can depend on the desired read depth for sequenced regions. Exemplary volumes are 0.4-40 ml, 5-20 ml, 10-20 ml. For example, the volume can be 0.5 ml, 1 ml, 5 ml, 10 ml, 20 ml, 30 ml, or 40 ml. A volume of sampled plasma may be 5 to 20 ml.

The sample can comprise various amounts of nucleic acid that contains genome equivalents. For example, a sample of about 30 ng DNA can contain about 10,000 (10⁴) haploid human genome equivalents and, in the case of cfDNA, about 200 billion (2×10¹¹) individual polynucleotide molecules. Similarly, a sample of about 100 ng of DNA can contain about 30,000 haploid human genome equivalents and, in the case of cfDNA, about 600 billion individual molecules.

A sample can comprise nucleic acids from different sources, e.g., from cells and cell free. A sample can comprise nucleic acids carrying mutations. For example, a sample can comprise DNA carrying germline mutations and/or somatic mutations. A sample can comprise DNA carrying cancer-associated mutations (e.g., cancer-associated somatic mutations).

Exemplary amounts of cell free nucleic acids in a sample before amplification range from about 1 fg to about 1 μg, e.g., 1 pg to 200 ng, 1 ng to 100 ng, 10 ng to 1000 ng. For example, the amount can be up to about 600 ng, up to about 500 ng, up to about 400 ng, up to about 300 ng, up to about 200 ng, up to about 100 ng, up to about 50 ng, or up to about 20 ng of cell-free nucleic acid molecules. The amount can be at least 1 fg, at least 10 fg, at least 100 fg, at least 1 pg, at least 10 pg, at least 100 pg, at least 1 ng, at least 10 ng, at least 100 ng, at least 150 ng, or at least 200 ng of cell-free nucleic acid molecules. The amount can be up to 1 femtogram (fg), 10 fg, 100 fg, 1 picogram (pg), 10 pg, 100 pg, 1 ng, 10 ng, 100 ng, 150 ng, or 200 ng of cell-free nucleic acid molecules. The method can comprise obtaining 1 femtogram (fg) to 200 ng.

A cell-free nucleic acid sample refers to a sample containing cell-free nucleic acids. In some embodiments, “cell-free nucleic acids” refers to nucleic acids not contained within or otherwise bound to a cell at the point of isolation from the subject. Cell-free nucleic acids can be referred to all non-encapsulated nucleic acid sourced from a bodily fluid (e.g., blood, urine, CSF, etc.) from a subject. Cell-free nucleic acids include DNA (cfDNA), RNA (cfRNA), and hybrids thereof, including genomic DNA, mitochondrial DNA, circulating DNA, siRNA, miRNA, circulating RNA (cRNA), tRNA, rRNA, small nucleolar RNA (snoRNA), Piwi-interacting RNA (piRNA), long non-coding RNA (long ncRNA), or fragments of any of these. Cell-free nucleic acids can be double-stranded, single-stranded, or a hybrid thereof. A cell-free nucleic acid can be released into bodily fluid through secretion or cell death processes, e.g., cellular necrosis and apoptosis. Some cell-free nucleic acids are released into bodily fluid from cancer cells e.g., circulating tumor DNA (ctDNA). Others are released from healthy cells. ctDNA can be non-encapsulated tumor-derived fragmented DNA. Cell-free fetal DNA (cffDNA) is fetal DNA circulating freely in the maternal blood stream.

A cell-free nucleic acid or proteins associated with it can have one or more epigenetic modifications, for example, a cell-free nucleic acid can be acetylated, 5-methylated, ubiquitylated, phosphorylated, sumoylated, ribosylated, and/or citrullinated.

Cell-free nucleic acids have an exemplary size distribution of about 100-500 nucleotides, with molecules of 110 to about 230 nucleotides representing about 90% of molecules, with a mode of about 168 nucleotides in humans and a second minor peak in a range between 240 to 430 nucleotides. Cell-free nucleic acids can be about 160 to about 180 nucleotides, or about 320 to about 360 nucleotides, or about 430 to about 480 nucleotides.

Cell-free nucleic acids can be isolated from bodily fluids through a partitioning step in which cell-free nucleic acids, as found in solution, are separated from intact cells and other non-soluble components of the bodily fluid. Partitioning may include techniques such as centrifugation or filtration. Alternatively, cells in bodily fluids can be lysed and cell-free and cellular nucleic acids processed together. Generally, after addition of buffers and wash steps, cell-free nucleic acids can be precipitated with an alcohol. Further clean up steps may be used such as silica based columns to remove contaminants or salts. Non-specific bulk carrier nucleic acids, for example, may be added throughout the reaction to optimize certain aspects of the procedure such as yield.

After such processing, samples can include various forms of nucleic acid including double-stranded DNA, single stranded DNA and single stranded RNA. Optionally, single stranded DNA and RNA can be converted to double-stranded forms so they are included in subsequent processing and analysis steps.

### Amplification

Sample nucleic acids flanked by adapters can be amplified by PCR and other amplification methods typically primed from primers binding to primer binding sites in adapters flanking a DNA molecule to be amplified. Amplification methods can involve cycles of extension, denaturation and annealing resulting from thermocycling or can be isothermal as in transcription mediated amplification. Other amplification methods include the ligase chain reaction, strand displacement amplification, nucleic acid sequence based amplification, and self-sustained sequence based replication.

One or more amplifications can be applied to introduce barcodes to a nucleic acid molecule using conventional nucleic acid amplification methods. The amplification can be conducted in one or more reaction mixtures. Molecular barcodes and sample indexes can be introduced simultaneously, or in any sequential order. Molecular barcodes and sample indexes can be introduced prior to and/or after sequence capturing. In some cases, only the molecular barcodes are introduced prior to probe capturing while the sample indexes are introduced after sequence capturing. In some cases, both the molecular barcodes and the sample indexes are introduced prior to probe capturing. In some cases, the sample indexes are introduced after sequence capturing. Usually, sequence capturing involves introducing a single-stranded nucleic acid molecule complementary to a targeted sequence, e.g., a coding sequence of a genomic region and mutation of such region is associated with a cancer type. Typically, the amplifications generate a plurality of non-uniquely or uniquely tagged nucleic acid amplicons with molecular barcodes and sample indexes at a size ranging from 200 nt to 700 nt, 250 nt to 350 nt, or 320 nt to 550 nt. In some implementations, the amplicons have a size of about 300 nt. In some implementations, the amplicons have a size of about 500 nt.

### Barcodes

Barcodes can be incorporated into or otherwise joined to adapters by chemical synthesis, ligation, overlap extension PCR among other methods. Generally, assignment of unique or non-unique barcodes in reactions follows methods and systems described by US patent applications 20010053519, 20110160078, and U.S. Pat. Nos. 6,582,908 and 7,537,898 and 9,598,731.

Tags can be linked to sample nucleic acids randomly or non-randomly. In some cases, they are introduced at an expected ratio of identifiers (e.g., a combination of barcodes) to microwells. The collection of barcodes can be unique, e.g., all the barcodes have different nucleotide sequence. The collection of barcodes can be non-unique, e.g., some of the barcodes have the same nucleotide sequence, and some of the barcodes have different nucleotide sequence. For example, the identifiers may be loaded so that more than 1, 2, 3, 4, 5, 6, 7, 8, 9, 10, 20, 50, 100, 500, 1000, 5000, 10000, 50,000, 100,000, 500,000, 1,000,000, 10,000,000, 50,000,000 or 1,000,000,000 identifiers are loaded per genome sample. In some cases, the identifiers may be loaded so that less than 2, 3, 4, 5, 6, 7, 8, 9, 10, 20, 50, 100, 500, 1000, 5000, 10000, 50,000, 100,000, 500,000, 1,000,000, 10,000,000, 50,000,000 or 1,000,000,000 identifiers are loaded per genome sample. In some cases, the average number of identifiers loaded per sample genome is less than, or greater than, about 1, 2, 3, 4, 5, 6, 7, 8, 9, 10, 20, 50, 100, 500, 1000, 5000, 10000, 50,000, 100,000, 500,000, 1,000,000, 10,000,000, 50,000,000 or 1,000,000,000 identifiers per genome sample.

A preferred format uses 20-50 different tags, ligated to both ends of a target molecule creating 20-50×20-50 tags, e.g., 400-2500 tags combinations. Such numbers of tags are sufficient that different molecules having the same start and stop points have a high probability (e.g., at least 94%, 99.5%, 99.99%, 99.999%) of receiving different combinations of tags.

In some cases, identifiers may be predetermined or random or semi-random sequence oligonucleotides. In other cases, a plurality of barcodes may be used such that barcodes are not necessarily unique to one another in the plurality. In this example, barcodes may be attached (e.g., by ligation or PCR amplification) to individual molecules such that the combination of the barcode and the sequence it may be attached to creates a unique sequence that may be individually tracked. As described herein, detection of non-uniquely tagged barcodes in combination with sequence data of beginning (start) and end (stop) positions of sequence reads may allow assignment of a unique identity to a particular molecule. The length, or number of base pairs, of an individual sequence read may also be used to assign a unique identity to such a molecule. As described herein, fragments from a single strand of nucleic acid having been assigned a unique identity, may thereby permit subsequent identification of fragments from the parent strand, and/or a complementary strand.

### Sequencing Pipeline

Sample nucleic acids flanked by adapters with or without prior amplification can be subject to sequencing, such as by one or more sequencing devices 107. Sequencing methods include, for example, Sanger sequencing, high-throughput sequencing, pyrosequencing, sequencing-by-synthesis, single-molecule sequencing, nanopore sequencing, semiconductor sequencing, sequencing-by-ligation, sequencing-by-hybridization, RNA-Seq (Illumina), Digital Gene Expression (Helicos), Next generation sequencing, Single Molecule Sequencing by Synthesis (SMSS) (Helicos), massively-parallel sequencing, Clonal Single Molecule Array (Solexa), shotgun sequencing, Ion Torrent, Oxford Nanopore, Roche Genia, Maxim-Gilbert sequencing, primer walking, sequencing using PacBio, SOLiD, Ion Torrent, or Nanopore platforms. Sequencing reactions can be performed in a variety of sample processing units, which may be multiple lanes, multiple channels, multiple wells, or other means of processing multiple sample sets substantially simultaneously. Sample processing unit can also include multiple sample chambers to enable processing of multiple runs simultaneously.

The sequencing reactions can be performed on one or more fragments types known to contain markers of cancer of other disease. The sequencing reactions can also be performed on any nucleic acid fragments present in the sample. The sequence reactions may provide for sequence coverage of the genome of at least 5%, 10%, 15%, 20%, 25%, 30%, 40%, 50%, 60%, 70%, 80%, 90%, 95%, 99%, 99.9% or 100%. In other cases, sequence coverage of the genome may be less than 5%, 10%, 15%, 20%, 25%, 30%, 40%, 50%, 60%, 70%, 80%, 90%, 95%, 99%, 99.9% or 100%.

Simultaneous sequencing reactions may be performed using multiplex sequencing. In some cases, cell free polynucleotides may be sequenced with at least 1000, 2000, 3000, 4000, 5000, 6000, 7000, 8000, 9000, 10000, 50000, 100,000 sequencing reactions. In other cases, cell free polynucleotides may be sequenced with less than 1000, 2000, 3000, 4000, 5000, 6000, 7000, 8000, 9000, 10000, 50000, 100,000 sequencing reactions. Sequencing reactions may be performed sequentially or simultaneously. Subsequent data analysis may be performed on all or part of the sequencing reactions. In some cases, data analysis may be performed on at least 1000, 2000, 3000, 4000, 5000, 6000, 7000, 8000, 9000, 10000, 50000, 100,000 sequencing reactions. In other cases, data analysis may be performed on less than 1000, 2000, 3000, 4000, 5000, 6000, 7000, 8000, 9000, 10000, 50000, 100,000 sequencing reactions. An exemplary read depth is 1000-50000 reads per locus (base).

Sequence Analysis Pipeline

The present methods can be used to diagnose presence of conditions, particularly cancer, in a subject, to characterize conditions (e.g., staging cancer or determining heterogeneity of a cancer), monitor response to treatment of a condition, effect prognosis risk of developing a condition or subsequent course of a condition.

Various cancers may be detected using the present methods. Cancers cells, as most cells, can be characterized by a rate of turnover, in which old cells die and replaced by newer cells. Generally dead cells, in contact with vasculature in a given subject, may release DNA or fragments of DNA into the blood stream. This is also true of cancer cells during various stages of the disease. Cancer cells may also be characterized, dependent on the stage of the disease, by various genetic aberrations such as copy number variation as well as rare mutations. This phenomenon may be used to detect the presence or absence of cancers individuals using the methods and systems described herein.

The types and number of cancers that may be detected may include blood cancers, brain cancers, lung cancers, skin cancers, nose cancers, throat cancers, liver cancers, bone cancers, lymphomas, pancreatic cancers, skin cancers, bowel cancers, rectal cancers, thyroid cancers, bladder cancers, kidney cancers, mouth cancers, stomach cancers, solid state tumors, heterogeneous tumors, homogenous tumors, and the like.

Cancers can be detected from genetic variations including mutations, rare mutations, indels, copy number variations, transversions, translocations, inversion, deletions, aneuploidy, partial aneuploidy, polyploidy, chromosomal instability, chromosomal structure alterations, gene fusions, chromosome fusions, gene truncations, gene amplification, gene duplications, chromosomal lesions, DNA lesions, abnormal changes in nucleic acid chemical modifications, abnormal changes in epigenetic patterns.

Genetic data can also be used for characterizing a specific form of cancer. Cancers are often heterogeneous in both composition and staging. Genetic profile data may allow characterization of specific sub-types of cancer that may be important in the diagnosis or treatment of that specific sub-type. This information may also provide a subject or practitioner clues regarding the prognosis of a specific type of cancer and allow either a subject or practitioner to adapt treatment options in accord with the progress of the disease. Some cancers progress, becoming more aggressive and genetically unstable. Other cancers may remain benign, inactive, or dormant. The system and methods of this disclosure may be useful in determining disease progression.

The present analysis is also useful in determining the efficacy of a particular treatment option. Successful treatment options may increase the amount of copy number variation or rare mutations detected in a subject's blood if the treatment is successful as more cancers may die and shed DNA. In other examples, this may not occur. In another example, perhaps certain treatment options may be correlated with genetic profiles of cancers over time. This correlation may be useful in selecting a therapy. Additionally, if a cancer is observed to be in remission after treatment, the present methods can be used to monitor residual disease or recurrence of disease.

The present methods can also be used for detecting genetic variations in conditions other than cancer. Immune cells, such as B cells, may undergo rapid clonal expansion upon the presence certain diseases. Clonal expansions may be monitored using copy number variation detection and certain immune states may be monitored. In this example, copy number variation analysis may be performed over time to produce a profile of how a particular disease may be progressing. Copy number variation or even rare mutation detection may be used to determine how a population of pathogens are changing during the course of infection. This may be particularly important during chronic infections, such as HIV/AIDs or Hepatitis infections, whereby viruses may change life cycle state and/or mutate into more virulent forms during the course of infection. The present methods may be used to determine or profile rejection activities of the host body, as immune cells attempt to destroy transplanted tissue to monitor the status of transplanted tissue as well as altering the course of treatment or prevention of rejection.

### Precision Treatment Example

In some embodiments, a subject having copy number loss that results in a loss of function, such as copy number loss relating to the human leukocyte antigen (HLA), may be administered a targeted therapy. In some cases, the targeted therapy may comprise an immunotherapy. The majority of cancer immunotherapies, including immune checkpoint blockade therapy, aim to counteract immune evasion by shifting the balance in favor of immune activation, enabling T cell-mediated cancer cell elimination. The key step shared by these therapies requires T cells to recognize the specific peptides presented by the HLAs on the membranes of tumor cells and subsequently kick off an immune response. Copy number loss in the HLA locus may result in tumor cells acquiring resistance through immune evasion, making immunotherapies less effective.

As described in “Research Status and Outlook of PD-1/PD-L1 Inhibitors for Cancer Therapy” by Ai, et. al. (Drug Des Devel Ther. 2020; 14: 3625-3649), the content of which is incorporated by reference in its entirety herein, the FDA has three immune checkpoint inhibitors for the Programmed cell Death (“PD”) PD-1 pathway: pembrolizumab, nivolumab and cemiplimab and three immune checkpoint inhibitors for the PD-L1 pathway: atezolizumab, avelumab and durvalumab.

In some cases, the targeted therapy may comprise poly ADP ribose polymerase inhibitors (PARPi), which are a group of pharmacological inhibitors of the enzyme poly ADP ribose polymerase (PARP). PARP inhibitors have been developed for multiple indications, including breast, ovarian, prostate, and pancreatic cancers. Patients with loss of function mutations, including copy number loss, in tumor suppressor genes, such as BRCA1, BRCA2, and PALB2 are good candidates for PARPi treatment. In certain embodiments, PARPi inhibitors that may be administered include olaparib, niraparib, or rucaparib. In some embodiments, the targeted therapy may comprise a combination therapy, for example, administering an immune checkpoint inhibitor and a PARPi.

As described by Hwang et al., in “Targeting loss of heterozygosity for cancer-specific immunotherapy” (Proc Natl Acad Sci USA. 2021 Mar. 23; 118(12): e2022410118.), the content of which is incorporated by reference in its entirety herein, NASCAR (Neoplasm-targeting Allele-Sensing CAR) is a therapeutic approach to target HLA alleles and can, in theory, extend to LOH of other polymorphic genes that result in altered cell surface antigens in cancers. This form of T cell engineering deploys a NOT-gate Boolean logic system comprising a chimeric antigen receptor (CAR) targeting the allele of human leukocyte antigen (HLA) that is retained in the cancer cells and an inhibitory CAR (iCAR) targeting the HLA allele that is lost in the cancer cells.

The precision diagnostics provided by the improved computer system 110 may result in precision treatment plans, which may be identified by the computer system 110 (and/or curated by health professionals). For example, it has been shown that HLA gene expression and mutation is associated with patient response to immune checkpoint blockade. Schaafsma, E., Fugle, C. M., Wang, X. et al. Pan-cancer association of HLA gene expression with cancer prognosis and immunotherapy efficacy. Br J Cancer 125, 422-432 (2021), the content of which is incorporated by reference in its entirety herein. It has also been shown that HLA-1 genotype, in particular loss of heterozygosity (LOH) may affect survivability and response to immune checkpoint blockade. Chowell, D. et al. Patient HLA class I (HLA-A, HLA-B, and/or HLA-C) genotype influences cancer response to checkpoint blockade immunotherapy. Science Vol. 359, Issue 6375, pp. 582-587 (2021), the content of which is incorporated by reference in its entirety herein.

Nucleotide variations in sequenced nucleic acids can be determined by comparing sequenced nucleic acids with a reference sequence. The reference sequence is often a known sequence, e.g., a known whole or partial genome sequence from an object, whole genome sequence of a human object. The reference sequence can be hG19. The sequenced nucleic acids can represent sequences determined directly for a nucleic acid in a sample, or a consensus of sequences of amplification products of such a nucleic acid, as described above. A comparison can be performed at one or more designated positions on a reference sequence. A subset of sequenced nucleic acids can be identified including a position corresponding with a designated position of the reference sequence when the respective sequences are maximally aligned. Within such a subset it can be determined which, if any, sequenced nucleic acids include a nucleotide variation at the designated position, and optionally which if any, include a reference nucleotide (i.e., same as in the reference sequence). If the number of sequenced nucleic acids in the subset including a nucleotide variant exceeds a threshold, then a variant nucleotide can be called at the designated position. The threshold can be a simple number, such as at least 1, 2, 3, 4, 5, 6, 7, 8, 9, or 10 sequenced nucleic acid within the subset including the nucleotide variant or it can be a ratio, such as a least 0.5, 1, 2, 3, 4, 5, 10, 15, or 20 of sequenced nucleic acids within the subset include the nucleotide variant, among other possibilities. The comparison can be repeated for any designated position of interest in the reference sequence. Sometimes a comparison can be performed for designated positions occupying at least 20, 100, 200, or 300 contiguous positions on a reference sequence, e.g., 20-500, or 50-300 contiguous positions.

All patent filings, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, implementation, or aspect of the disclosure can be used in combination with any other unless specifically indicated otherwise. Although the present disclosure has been described in some detail by way of illustration and example for purposes of clarity and understanding, it will be apparent that certain changes and modifications may be practiced within the scope of the appended claims.

1. A computer system to determine a copy number variant (CNV) at a locus of interest in a sample of a subject when direct measurements of copy number at the locus of interest are unavailable, the computer system comprising:
a processor programmed to:
access a plurality of sequence reads of a sample;
map the plurality of sequence reads to a reference sequence, the reference sequence including a sequence of interest that includes the locus of interest;
generate a copy number score indicating a copy number of regions along the sequence of interest, the copy number score not including a copy number at the locus of interest;
generate a plurality of segments along the sequence of interest;
for each segment of the plurality of segments:
generate a respective segmented copy number score based on the segment and the copy number score;
generate a respective weight based on historical performance of the segment in predicting a CNV at the locus of interest;
generate a weighted score based on the respective weight and the respective segmented copy number score; and
apply a probabilistic machine-learning (ML) copy number classifier to generate a probability of the CNV at the locus of interest based on an aggregate of the weighted score of each segment.

2. The computer system of claim 1, wherein the processor is further programmed to:
train the probabilistic ML copy number classifier based on a maximum likelihood estimation.

3. The computer system of claim 1, wherein the processor is further programmed to:
train the probabilistic ML copy number classifier based on a Hidden Markov Model.

4. The computer system of claim 1, wherein the processor is further programmed to:
train the probabilistic ML copy number classifier based on Bayesian inference.

5. A computer system to determine a genetic state of a locus of interest of a genetic material in a sample of a subject, comprising:
a processor programmed to:
detect a genetic state associated with a segment in the genetic material in the sample;
generate a probability that the locus of interest outside the segment is also in the genetic state; and
predict a disease condition of the subject based on the generated probability.

6. The computer system of claim 5, wherein the genetic material comprises cell free DNA derived from the locus of interest and wherein the genetic state comprises a copy number variant at the segment of the chromosome.

7. The computer system of claim 6, wherein to generate the probability, the processor is further programmed to:
determine a level of deletion of the chromosome based on the copy number variant at the segment, wherein the generated probability rises as a proportional function with the level of deletion.

8. The computer system of claim 6, wherein to generate the probability, the processor is further programmed to:
determine a physical distance between the segment and the locus of interest, wherein the generated probability rises as an inversely proportional function with the physical distance.

9. The computer system of claim 5, wherein to detect the genetic state, the processor is programmed to:
access sequence reads generated from the sample; and
analyze sequence coverage information of the sequence reads.

10. The computer system of claim 5, wherein to generate the probability, the processor is programmed to generate the probability without sequence reads that cover the locus of interest.

11. The computer system of claim 5, wherein to generate the probability, the processor is programmed to generate the probability without genotyping data for the locus of interest.

12. The computer system of claim 5, wherein the locus of interest is associated with the disease condition.

13. The computer system of claim 12, wherein the locus of interest comprises a Human Leukocyte Antigens (HLA) locus.

14. The computer system of claim 12, one or more target genes in the locus of interest and one or more reference genes in the segment are not within the same gene regulation pathway.

15. The computer system of claim 5, wherein one or more target genes in the locus of interest are in genetic linkage with one or more reference genes in the segment, and wherein the processor is programmed to identify one or more segments, including the segment, to analyze for detection of copy number variants based on the genetic linkage.

16. The computer system of claim 5, wherein the genetic state comprises at least one of: a copy number variation, an aneuploidy, a segmental loss, a fusion, or an indel.

17. The computer system of claim 5, wherein the processor is further programmed to:
determine a treatment based on the predicted disease condition.

18. The computer system of claim 5, wherein to detect the genetic state, the processor is further programmed to:
determine that the segment includes a copy number loss based on probe data for probes directed to the segment; and
wherein to generate a probability that the locus of interest outside the segment is also in the genetic state, the processor is further programmed to determine that the locus of interest also includes a copy number loss.

19.-45. (canceled)
